(19) United States
(12) Patent Application Publication
(10) Pub. No.: US 2020/0011858 A1
(43) Pub. Date: Jan. 9, 2020

(54) ELECTRODE DEVICE FOR ANALYZING BIOMATERIAL

(71) Applicant: BIOMEDUX CO., LTD, Suwon-si (KR)

(72) Inventors: Jong Won KIM, Seoul (KR); Ji Hyoung LEE, Seoul (KR); Sang Yoon KIM, Chuncheon-si (KR); Ah Reum PARK, Seoul (KR); In Young KIM, Pyeongtaek-si (KR); Doo Hee JEONG, Suwon-si (KR)

(21) Appl. No.: 16/490,495

(22) PCT Filed: May 17, 2017
(86) PCT No.: PCT/KR2017/005088

§ 371 Date: Aug. 30, 2019

(30) Foreign Application Priority Data
Apr. 13, 2017 (KR) 10-2017-0048029

(51) Int. Cl. G01N 33/543; G01N 27/04; G01N 27/42; G01N 27/327; H05K 1/18; G01N 33/487

(52) U.S. Cl. CPC G01N 33/543; G01N 27/04; G01N 33/48707; G01N 27/327; H05K 1/18; G01N 27/42

(57) ABSTRACT

Provided is an electrode device for analyzing a biomaterial, which includes: an electrode portion having a plurality of electrodes arranged spaced apart from a plate-shaped substrate and a biomaterial input portion formed on the substrate to be electrically connected to at least one of the plurality of electrodes; a housing having an electrode accommodation portion having one side open and accommodating the electrode portion; a printed circuit board fixed to the housing; a connector portion formed on one end portion of the printed circuit board and electrically and detachably coupled to an analysis apparatus; connection pillars electrically connected to the connector portion and having one end portion fixed to the printed circuit board and the other end portion formed to be in pressure contact with the electrode portion, wherein the connection pillars includes an electrically conductive material and is arranged to correspond to the plurality of electrodes; and an input hole penetrating through an upper surface and a lower surface of the printed circuit board to be in communication with the electrode portion.

### TECHNICAL FIELD

The present disclosure relates to an electrode device detachably coupled to a biomaterial analyzing apparatus, wherein a biomaterial is injected into the electrode device.

### BACKGROUND ART

Human genetic information has been known accurately and precisely with the development of modern medicine and biology. Accordingly, more information on DNA, RNA, proteins, etc. related to diseases is being revealed.

In order to diagnose diseases such as cancer early, it is necessary to sensitively detect a very small amount of a biomaterial, such as proteins and DNA, associated with diseases, from a patient's blood or body fluids, at an early stage of disease.

With the development of electronics and genetic engineering, there is a growing tendency for electronic technology to be integrated into the detection of biomaterials. Typically, the detection of biomaterials involves accommodating a small amount of a biomaterial in a biochip and obtaining information about the biomaterial in an optical or electrochemical method. Korean Patent No. 1218987 discloses an example of a biochip technology for detection of biomaterials.

In an optical method according to the related art, light is irradiated to a biomaterial and fluorescence generated from the biomaterial is analyzed. Such an optical method requires a labeling of the biomaterial as a basis for fluorescence analysis, and thus, the method is inconvenient and the cost of analysis is expensive.

Meanwhile, in the electrochemical method as disclosed in Korean Patent No. 1218987, information on a biomaterial is obtained by analyzing a signal obtained by passing an electric signal generated by a direct digital synthesis using electronic engineering technology and a lock-in detection technique using a trans-impedance amplifier through the biomaterial.

However, the biochip according to the related art disclosed in the above patent includes a reaction portion for allowing a biomaterial to react and a connector electrically connected to the reaction portion, which are provided on a glass substrate by being plated with a material such as gold or copper. The gold or copper plated on the glass substrate has a weak adhesive force. Accordingly, the connector may be easily damaged by a repeated use of a biochip. In particular, the connector of a biochip may be rapidly abraded because it receives a physically great frictional force in a process of being coupled to a socket of an electronic apparatus for analysis.

### DESCRIPTION OF EMBODIMENTS

### Technical Problem

Provided is an electrode device for analyzing a biomaterial, which has remarkably enhanced durability even in repeated use thereof by improving the structure of an electrode device for analysis of a biomaterial.

### Solution to Problem

According to an aspect of the present disclosure, an electrode device for analyzing a biomaterial includes: an electrode portion having a plurality of electrodes arranged spaced apart from a plate-shaped substrate and a biomaterial input portion formed on the substrate to be electrically connected to at least one of the plurality of electrodes;

a housing having an electrode accommodation portion having one side open and accommodating the electrode portion;

a printed circuit board fixed to the housing;

a connector portion formed on one end portion of the printed circuit board and electrically and detachably coupled to an analysis apparatus;

connection pillars electrically connected to the connector portion and having one end portion fixed to the printed circuit board and the other end portion formed to be in pressure contact with the electrode portion, wherein the connection pillars includes an electrically conductive material and is arranged to correspond to the plurality of electrodes; and

an input hole penetrating through an upper surface and a lower surface of the printed circuit board to be in communication with the electrode portion.

### Advantageous Effects of Disclosure

In the electrode device for analyzing a biomaterial according to the present disclosure, as the electrode portion to which a biomaterial is input and the connector portion repeatedly attached to or detached from the analysis apparatus are separately provided, the durability of the connector portion is reinforced and improved, thereby remarkably improving the life of the electrode device. Furthermore, in the electrode device for analyzing a biomaterial according to the present disclosure, as the electrode portion is replaceable, only the electrode portion may be manufactured for replacement at an economical cost and the manufacturing cost of the electrode device may be reduced. Furthermore, in the electrode device for analyzing a biomaterial according to the present disclosure, as the input hole is provided in the printed circuit board, a biomaterial may be easily input to or removed from the electrode portion, and thus reuse of the electrode portion is available.

### BRIEF DESCRIPTION OF DRAWINGS

FIG. 1 is a perspective view of an electrode device for analyzing a biomaterial according to an embodiment of the present disclosure.

FIG. 2 is an exploded perspective view of the electrode device illustrated in FIG. 1.

FIG. 3 is a cross-sectional view taken along line III-III of FIG. 1.

FIG. 4 illustrates a detailed structure of an electrode portion illustrated in FIG. 2.

FIG. 5 illustrates a detailed structure of a housing illustrated in FIG. 2.

FIG. 6 illustrates a detailed arrangement structure of a connection pillar illustrated in FIG. 2.

FIG. 7 illustrates a state in which the electrode device illustrated in FIG. 1 is coupled to an SD card slot.

### BEST MODE

Hereinafter, an embodiment according to the present disclosure is described in detail with reference to the accompanying drawings.

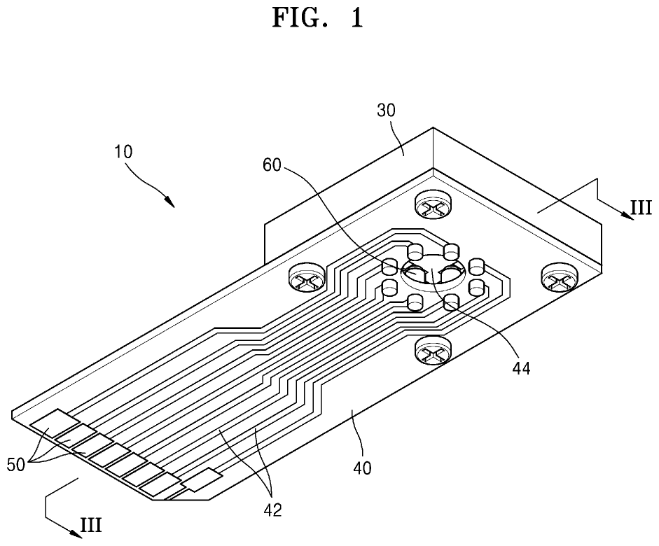

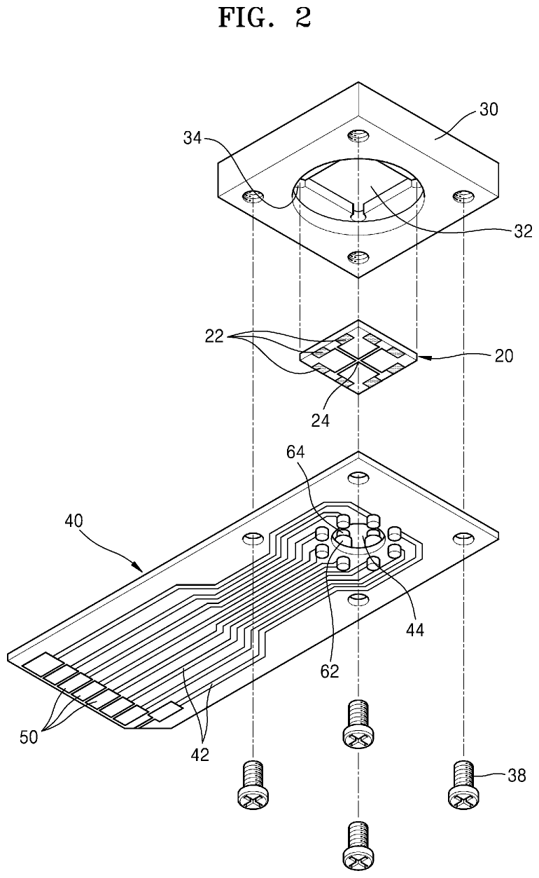

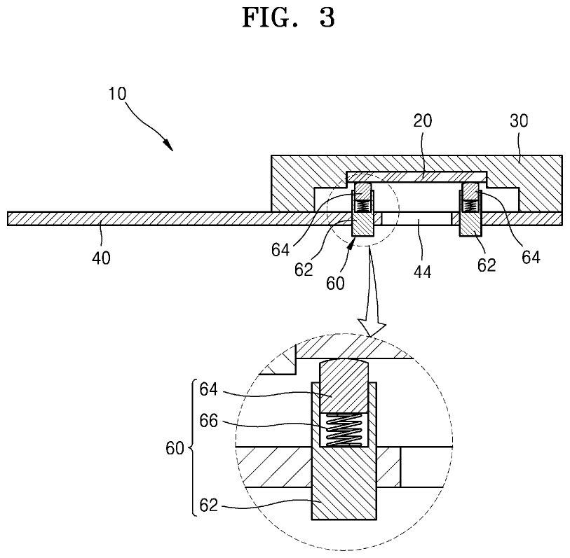

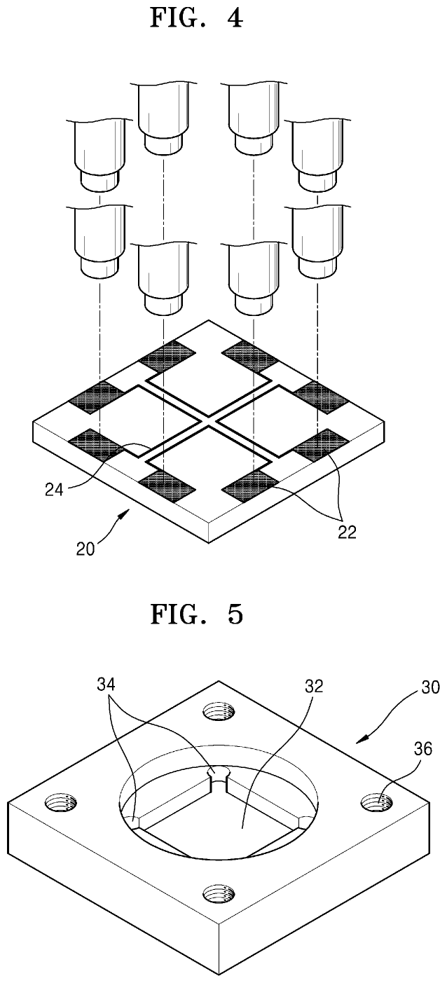

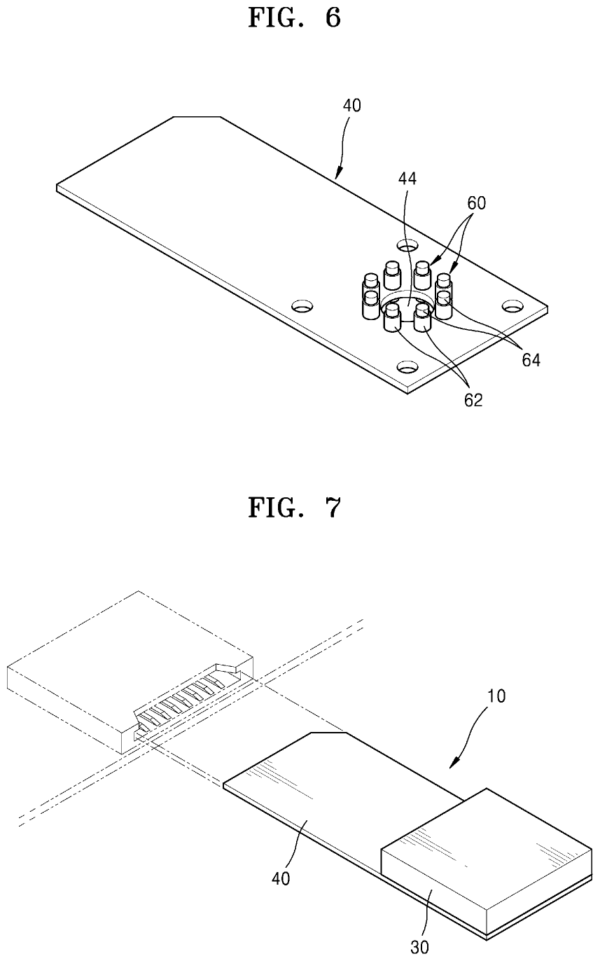

FIG. 1 is a perspective view of an electrode device for analyzing a biomaterial according to an embodiment of the present disclosure. FIG. 2 is an exploded perspective view of the electrode device illustrated in FIG. 1. FIG. 3 is a cross-sectional view taken along line III-III of FIG. 1. FIG. 4 illustrates a detailed structure of an electrode portion illustrated in FIG. 2. FIG. 5 illustrates a detailed structure of a housing illustrated in FIG. 2. FIG. 6 illustrates a detailed arrangement structure of a connection pillar illustrated in FIG. 2. FIG. 7 illustrates a state in which the electrode device illustrated in FIG. 1 is coupled to an SD card slot.

Referring to FIGS. 1 to 7, an electrode device for analyzing a biomaterial according to an embodiment of the present disclosure (10, hereinafter referred to as the “electrode device”) is a kind of a biosensor detachably coupled to a biomaterial analysis apparatus according to an electrochemical method.

The electrode device 10 may include an electrode portion 20, a housing 30, a printed circuit board 40, a connector portion 50, a connection pillar 60, and an input hole 44.

The electrode portion 20 is provided as a plate-shaped substrate. The electrode portion 20 may include a plurality of electrodes 22. The electrodes 22 are arranged spaced apart from each other. In the present embodiment, the electrodes 22 are arranged circularly.

In the present embodiment, the electrode portion 20 is provided as a square substrate. The electrode portion 20 may include two (2) electrodes along each side of the substrate. Accordingly, in the present embodiment, the electrode 22 may include eight (8) electrodes.

The electrode portion 20 may include a biomaterial input portion 24. The biomaterial input portion 24 is disposed to be electrically connected to at least one of the electrodes. In the present embodiment, the biomaterial input portion 24 is disposed at the center of the substrate. The biomaterial input portion 24 may be disposed at a position deviated to one side from the center of the substrate. The biomaterial input portion 24 is where a small amount of a biomaterial such as DNA in units of microliters (pQ) is input. The biomaterial input to the biomaterial input portion 24 generates an output pulse by reacting to an electric pulse signal input through the connector portion 50 that will be described below. The electric pulse signal input to the connector portion 50 is an analog voltage pulse signal generated through a general-purpose digital signal converter such as NI-DAQ and a general-purpose signal processing module such as AD9837 that is a direct digital synthesis device.

The electric pulse signal passed through the biomaterial input portion 24 is generated as a current pulse signal, converted to a voltage signal by a trans-impedance amplifier that is a separately formed well-known signal processing device, and converted to a digital signal by NI-DAQ. A digital value converted by the NI-DAQ to a digital signal may be processed into numerical data or video data that a user may utilize through MATLAB that is a well-known mathematical calculation program.

The housing 30 is a member for accommodating and fixing the electrode portion 20.

The housing 30 may include an electrode accommodation portion 32 having one side open. The electrode portion 20 is accommodated in the electrode accommodation portion 32 by being inserted therein. The housing 30 may be manufactured with synthetic resin that is electrically non-conductive. The housing 30 may include a tool insertion portion 34. The tool insertion portion 34 is disposed adjacent to the electrode accommodation portion 32. The tool insertion portion 34 is a space where a tool may be inserted when the electrode portion 20 accommodated in the electrode accommodation portion 32 is separated. In the present embodiment, the electrode accommodation portion 32 is a concave recess portion having a square shape. Furthermore, the tool insertion portion 34 is formed at each corner of the electrode accommodation portion 32. The housing 30 may include a plurality of coupling holes 36 for coupling with the printed circuit board 40 that will be described below. A female screw portion is formed on an inner circumference surface of each of the coupling holes 36. In the present embodiment, the coupling holes 36 may include four (4) coupling holes.

The printed circuit board 40 is fixed to the housing 30. The printed circuit board 40 is a plate-shaped member that is long in one direction. The printed circuit board 40 is a member having a circuit line portion 42 formed on one surface of a body thereof that is electrically non-conductive. The circuit line portion 42 may be formed by a combination of plating and etching processes. The printed circuit board 40 is firmly fixed to the housing 30 as a coupling bolt 38 passes through the printed circuit board 40 and is screw-coupled to each of the coupling holes 36 formed in the housing 30.

The connector portion 50 is formed at one end portion of the printed circuit board 40. The connector portion 50 is electrically connected to the circuit line portion 42. The connector portion 50 is formed by plating a metal material exhibiting a superior electrical conductivity, such as copper or gold on a surface of the body of the printed circuit board 40. When the connector portion 50 is integrally formed with the printed circuit board 40, durability may be remarkably improved compared with a structure according to the related art in which a connector portion is formed on a glass substrate. The connector portion 50 is detachably and electrically coupled to a separate biomaterial analysis apparatus. The connector portion 50 may be compatible with an SD card slot. In general, an SD card slot is an interface structure that is widely used for electronic apparatuses, and thus, the supply of parts thereof is easy and a cost thereof is low.

The connection pillar 60 is electrically connected to the connector portion 50. One end portion of the connection pillar 60 is fixed to the printed circuit board 40. The other end portion of the connection pillar 60 is formed to be in pressure contact with the electrode portion 20. In detail, the connection pillar 60 is disposed to be pressed by the electrode 22 formed on the electrode portion 20. The connection pillar 60 is formed of a material that is electrically conductive. For example, the connection pillar 60 may be manufactured of copper, aluminium, gold, silver, etc. The connection pillar 60 is disposed to correspond to the electrode 22. In the present embodiment, the connection pillar 60 is disposed circularly to correspond to the arrangement of the electrode 22. The connection pillar 60 may include a fixed portion 62 and a moving portion 64.

The fixed portion 62 is electrically connected to the circuit line portion 42. Furthermore, the fixed portion 62 is mechanically fixed to the printed circuit board 40. The fixed portion 62 protrudes, in a cantilever form, toward the electrode portion 20 from the printed circuit board 40.

The moving portion 64 is slidably coupled to the fixed portion 62. The moving portion 64 coupled to the fixed portion 62 elastically presses the electrode 22. The fixed portion 62 and the moving portion 64 may be coupled to each other via an elastic member 66, for example, a coil spring. The moving portion 64 performs a function of maintaining the electric connection between the connector portion 50 and the electrode portion 20 always in a good condition. Furthermore, the moving portion 64 absorbs allowance generated in a process of manufacturing the connection pillar 60 and thus provides an operation and an effect of maintaining superior assembly quality of the electrode device 10.

The input hole 44 penetrates through an upper surface and a lower surface of the printed circuit board 40. The input hole 44 is in communication with the electrode portion 20. In detail, the input hole 44 is in communication with the biomaterial input portion 24. A user may input a small amount of a biomaterial to the electrode portion 20 or remove the biomaterial input to the electrode portion 20, through the input hole 44. A tool such as a pipette may be used for the input of a biomaterial. The biomaterial may be removed by using a tool such as a cotton swab.

In the following description, the operation and effect of the present disclosure are described in detail with an example of a method of using the electrode device 10 for analyzing a biomaterial including the above-described elements.

The concentration of DNA has been known to have a close correlation with impedance generated due to application of an electric pulse signal having a certain frequency value. A case of inputting a biomaterial such as DNA to an electrode device is described with reference to FIG. 1.

When a fine liquid biomaterial is input to an electrode device by using a pipette, a small amount of a biomaterial is input to the biomaterial input portion 24 through the input hole 44. The connector portion 50 is coupled to an analysis apparatus. As the connector portion 50 is compatible with an SD card slot, the connector portion 50 may be easily coupled to an analysis apparatus having an SD card slot. An analog pulse voltage signal having a specific frequency value is applied to the connector portion 50 through NI-DAQ that is a well-known electric signal processing apparatus and AD9837 that is a digital signal synthetic apparatus. The analog pulse voltage signal applied to the connector portion 50 is transmitted to the electrode portion 20 via the circuit line portion 42 and the connection pillar 60. The analog pulse voltage signal transmitted to the electrode portion 20 passes through the biomaterial input to the biomaterial input portion 24 and generates a pulse signal different from an input pulse due to impedance (resistance). An output pulse generated by the biomaterial input portion 24 islfeed-backed to the connector portion 50 via the connection pillar 60 and the circuit line portion 42. An electric pulse signal output through the connector portion 50 passes through the trans-impedance amplifier provided in the analysis apparatus and thus a current pulse signal is converted to a voltage pulse signal. The voltage pulse signal output from the trans-impedance amplifier, which is an analog signal, is input to the NI-DAQ that is a well-known signal processing apparatus and then is processed into a numeral or video data that may be easily understood by the user, by using a mathematical calculation program such as MATLAB.

In the above process, the electrode device 10 is configured such that the connector portion 50 electrically connected to the analysis apparatus is physically separated from the electrode portion 20 to which a biomaterial is input. Accordingly, as the electrode device 10 is repeatedly coupled to or separated from the analysis apparatus, a frictional force is repeatedly generated in the connector portion 50. However, as the connector portion 50 is formed on one end portion of the printed circuit board 40 formed of synthetic resin, durability may be remarkably improved compared with an electrode device structure according to the related art in which a connector portion is formed on a surface of a glass substrate. In other words, forming the connector portion on a surface of synthetic resin has higher strength and higher adhesive force than forming the connector portion on the glass substrate. Accordingly, during repeated use of the electrode device 10, durability may be improved compared with the electrode device according to the related art.

Furthermore, as the electrode device 10 according to the present disclosure is configured to replace only the electrode portion 20 that does not repeatedly contact the analysis apparatus, the manufacturing cost of the electrode portion 20 is low. Furthermore, the electrode device 10 according to the present disclosure is advantageous, compared with the structure according to the related art, in that the electrode device 10 may be used semi-permanently by replacing the electrode portion 20 only.

As such, in the electrode device for analyzing a biomaterial according to the present disclosure, as the electrode portion to which a biomaterial is input and the connector portion repeatedly attached to or detached from the analysis apparatus are separately provided, the durability of the connector portion is reinforced and improved, thereby remarkably improving the life of the electrode device. Furthermore, in the electrode device for analyzing a biomaterial according to the present disclosure, as the electrode portion is replaceable, only the electrode portion may be manufactured for replacement at an economical cost and the manufacturing cost of the electrode device may be reduced. Furthermore, in the electrode device for analyzing a biomaterial according to the present disclosure, as the input hole is provided in the printed circuit board, a biomaterial may be easily input to or removed from the electrode portion, and thus, reuse of the electrode portion is available.

While the present disclosure has been particularly shown and described with reference to preferred embodiments using specific terminologies, the embodiments and terminologies should be considered in descriptive sense only and not for purposes of limitation. Therefore, it will be understood by those of ordinary skill in the art that various changes in form and details may be made therein without departing from the spirit and scope of the present disclosure as defined by the following claims. MODE OF DISCLOSURE

According to an aspect of the present disclosure, an electrode device for analyzing a biomaterial includes: an electrode portion having a plurality of electrodes arranged spaced apart from a plate-shaped substrate and a biomaterial input portion formed on the substrate to be electrically connected to at least one of the plurality of electrodes;

a housing having an electrode accommodation portion having one side open and accommodating the electrode portion;

a printed circuit board fixed to the housing;

a connector portion formed on one end portion of the printed circuit board and electrically and detachably coupled to an analysis apparatus;

connection pillars electrically connected to the connector portion and having one end portion fixed to the printed circuit board and the other end portion formed to be in pressure contact with the electrode portion, wherein the connection pillars includes an electrically conductive material and is arranged to correspond to the plurality of electrodes; and

an input hole penetrating through an upper surface and a lower surface of the printed circuit board to be in communication with the electrode portion.

The plurality of electrodes may be circularly arranged spaced apart from each other, and the biomaterial input portion may be formed at a center of the substrate, and

the connection pillars may be circularly arranged to correspond to the plurality of electrodes.

Each of the connection pillars may include a portion fixed to the printed circuit board; and

each of the connection pillars may include a moving portion slidably coupled to the fixed portion and elastically pressed from the fixed portion toward the electrode.

The electrode device for analyzing a biomaterial may further include a tool insertion portion formed adjacent to the electrode accommodation portion, wherein a tool is inserted into the tool insertion portion when the electrode portion accommodated in the electrode accommodation portion is separated.

The connector portion may be compatible with an SD card slot.

The electrode portion may be provided by a square substrate and two electrodes may be disposed along each side of the substrate.

1. An electrode device for analyzing a biomaterial, the electrode device comprising:
an electrode portion having a plurality of electrodes arranged spaced apart from a plate-shaped substrate and a biomaterial input portion formed on the substrate to be electrically connected to at least one of the plurality of electrodes;
a housing having an electrode accommodation portion having one side open and accommodating the electrode portion;
a printed circuit board fixed to the housing;
a connector portion formed on one end portion of the printed circuit board and electrically and detachably coupled to an analysis apparatus;
connection pillars electrically connected to the connector portion and having one end portion fixed to the printed circuit board and the other end portion formed to be in pressure contact with the electrode portion, wherein the connection pillars includes an electrically conductive material and is arranged to correspond to the plurality of electrodes; and
an input hole penetrating through an upper surface and a lower surface of the printed circuit board to be in communication with the electrode portion.

2. The electrode device for analyzing a biomaterial of claim 1, wherein
the plurality of electrodes are circularly arranged spaced apart from each other, and the biomaterial input portion is formed at a center of the substrate, and
the connection pillars are circularly arranged to correspond to the plurality of electrodes.

3. The electrode device for analyzing a biomaterial of claim 1, wherein
each of the connection pillars comprises a portion fixed to the printed circuit board; and
each of the connection pillars comprises a moving portion slidably coupled to the fixed portion and elastically pressed from the fixed portion toward the electrode.

4. The electrode device for analyzing a biomaterial of claim 1, further comprising
a tool insertion portion formed adjacent to the electrode accommodation portion, wherein a tool is inserted into the tool insertion portion when the electrode portion accommodated in the electrode accommodation portion is separated.

5. The electrode device for analyzing a biomaterial of claim 1, wherein
the connector portion is compatible with an SD card slot.

6. The electrode device for analyzing a biomaterial of claim 1, wherein
the electrode portion is provided by a square substrate and two electrodes are disposed along each side of the substrate.
